(19) United States
(12) Patent Application Publication
(10) Pub. No.: US 2026/0116805 A1
(43) Pub. Date: Apr. 30, 2026

(54) HIGH-STRENGTH LOW-CREEP OVERFLOW BRICKS AND METHODS FOR DESIGNING THE SAME

(71) Applicant: CAIHONG DISPLAY DEVICES CO., LTD., Xianyang (CN)

(72) Inventors: Menghu LI, Xianyang (CN); Lihua XU, Xianyang (CN); Weidong HU, Xianyang (CN)

(73) Assignee: CAIHONG DISPLAY DEVICES CO., LTD., Xianyang (CN)

(21) Appl. No.: 19/393,630

(22) Filed: Nov. 19, 2025

Related U.S. Application Data
(63) Continuation-in-part of application No. PCT/CN2025/111259, filed on Jul. 29, 2025.

(30) Foreign Application Priority Data
Jul. 30, 2024 (CN) 202411033837.6

(51) Int. Cl. C03B 17/06; C03B 5/26

(52) U.S. Cl. CPC C03B 17/064; C03B 5/265

(57) ABSTRACT

A high-strength low-creep overflow brick and a method for designing the same are provided. The method includes: selecting a mature overflow brick for manufacturing a glass substrate as a reference overflow brick, obtaining design parameters of the reference overflow brick, design parameters of a designed overflow brick, and a creep stress exponent of the overflow brick; establishing similarity relationships between the reference and designed overflow bricks, and establishing a system of equations regarding a first height and a second height at an inlet cross-section of the designed overflow brick; determining strength parameters of the reference and designed overflow bricks based on the similarity relationships, the design parameters of the reference and designed overflow bricks, the system of equations; determining a support surface length of the designed overflow brick and a maximum cylinder pressure of a clamp cylinder based on the strength parameters of the reference and designed overflow bricks.

### CROSS-REFERENCE TO RELATED APPLICATIONS

This application is a continuation-in-part of International Application No. PCT/CN2025/111259, filed on Jul. 29, 2025, which claims priority to Chinese Patent Application No. 202411033837.6, filed on Jul. 30, 2024, the entire contents of each of which are hereby incorporated by reference.

### TECHNICAL FIELD

The present disclosure relates to the field of glass substrate manufacturing, and specifically to a high-strength low-creep overflow brick and a method for designing the same.

### BACKGROUND

In the field of precision manufacturing of Thin-Film Transistor Liquid Crystal Display (TFT-LCD) flat panel displays, the production of glass substrates relies on precise control of an overflow down-draw technique. The overflow down-draw technique involves feeding molten glass from a melting furnace to a fusion overflow down-draw forming apparatus to produce a glass substrate that meets stringent standards. Throughout this process, ensuring uniform thickness, low residual stress, and accurate warp control of the glass substrate represents a key challenge.

As a core component of the forming apparatus, the structural design of an overflow brick is particularly critical. With the evolution of product generations and increases in pull-out volume, higher optimization requirements are placed on the structure of an overflow system, especially on the structural design of the overflow brick, to ensure the stability of a glass drawing process.

When evaluating the performance of the overflow brick, it is necessary to comprehensively consider a chemical composition, a microstructure, a mechanical property, a thermal property, and high-temperature service performance of the overflow brick. In particular, a high-temperature flexural strength and a high-temperature creep are crucial metrics. The high-temperature flexural strength reflects a material's ability to resist bending stress in a high-temperature environment, and the high-temperature creep reflects time-dependent strain of the material under constant stress.

To precisely control a thickness of the glass substrate, the design of the overflow brick must fully account for the influence of various complex factors on a thickness distribution. These factors include, but are not limited to, a self-weight stress, a gravity of the molten glass, and a bending stress from a clamp cylinder. Furthermore, given a high-temperature operating environment (approximately 1,207° C.), it is essential to accurately calculate a creep rate under actual service conditions to reliably predict and control the impact of creep on the formed thickness. In this process, applying an appropriate cylinder pressure of the clamp cylinder is fundamental for effectively controlling the amount of creep, thereby ensuring the precision and stability of the glass substrate.

Therefore, designing an overflow brick with high strength and low creep characteristics, while simultaneously ensuring the cylinder pressure of the clamp cylinder remains within a reasonable range to meet the high-precision requirements for glass substrates in the manufacturing of TFT-LCD flat panel displays, has become a significant technical challenge urgently needing resolution by those skilled in the art.

### SUMMARY

One or more embodiments of the present disclosure provide a method for designing a high-strength low-creep overflow brick. The method includes: selecting a mature overflow brick for manufacturing a glass substrate as a reference overflow brick, obtaining design parameters of the reference overflow brick, and determining design parameters of a designed overflow brick; determining a proximal leg height H_{Iref} and a distal leg height H_{Oref} of the reference overflow brick based on the design parameters of the reference overflow brick; and determining a first average leg height H_{1ref} and a second average leg height H_{2ref} of the reference overflow brick based on the design parameters, the proximal leg height H_{Iref}, and the distal leg height H_{Oref} of the reference overflow brick. The method further includes: establishing similarity relationships between the reference overflow brick and the designed overflow brick, the similarity relationships including an overflow trough wall thickness similarity relationship, a first average leg height similarity relationship, and a second average leg height similarity relationship; determining an overflow trough wall thickness B of the designed overflow brick based on the overflow trough wall thickness similarity relationship; establishing a system of equations regarding a first height h₁ and a second height h₂ at an inlet cross-section of the designed overflow brick based on the design parameters, the proximal leg height H_{Iref}, the distal leg height H_{Oref}, the first average leg height H_{1ref}, and the second average leg height H_{2ref} of the reference overflow brick, the design parameters and the overflow trough wall thickness B of the designed overflow brick, and the similarity relationships between the reference overflow brick and the designed overflow brick, and determining the first height h₁ and the second height h₂ at the inlet cross-section of the designed overflow brick based on the system of equations. The method further includes: determining strength parameters of the reference overflow brick and the designed overflow brick based on the design parameters, the proximal leg height H_{Iref}, and the distal leg height H_{Oref} of the reference overflow brick, and the design parameters, the overflow trough wall thickness B, and the first height h₁ and the second height h₂ at the inlet cross-section of the designed overflow brick; and determining a support surface length l of the designed overflow brick and a maximum cylinder pressure Pₘₐₓ of a clamp cylinder based on the strength parameters of the reference overflow brick and the designed overflow brick to complete the design of the designed overflow brick.

In some embodiments, the proximal leg height H_{Iref} of the reference overflow brick is determined based on Equation (1):

$${H}_{Iref} = {h}_{1 ref} - [ \frac{{w}_{ref} + 2 \times {B}_{ref}}{2 \times tan \frac{{θ}_{ref}}{2}} - ( \frac{{R}_{ref}}{sin \frac{{θ}_{ref}}{2}} - {R}_{ref} ) - {h}_{2 ref} ] , \qquad ( 1 )$$

the distal leg height H_{Oref} of the reference overflow brick is determined based on Equation (2):

$${H}_{Oref} = {H}_{Iref} + {h}_{ref} - {W}_{ref} \times tan {ϕ}_{ref} . \qquad ( 2 )$$

In Equations (1) and (2), h_{1ref} denotes a first height at an inlet cross-section of the reference overflow brick, w_{ref} denotes an overflow surface width of the reference overflow brick, B_{ref} denotes an overflow trough wall thickness of the reference overflow brick, θ_{ref} denotes an overflow inclined surface angle of the reference overflow brick, R_{ref} denotes an overflow brick tip chamfer radius of the reference overflow brick, h_{2ref} denotes a second height at the inlet cross-section of the reference overflow brick, φ_{ref} denotes an overflow weir inclination angle of the reference overflow brick, and h_{ref} denotes an overflow trough depth at the inlet cross-section of the reference overflow brick.

In some embodiments, the first average leg height H_{1ref} of the reference overflow brick is determined based on Equation (3):

$${H}_{1 ref} = \frac{{H}_{Iref} + {H}_{Oref}}{2} , \qquad ( 3 )$$

the second average leg height H_{2ref} of the reference overflow brick is determine based on Equation (4):

$${H}_{2 ref} = {H}_{1 ref} + ( {h}_{1 ref} - {H}_{Iref} ) . \qquad ( 4 )$$

In some embodiments, the overflow trough wall thickness similarity relationship is represented by Equation (5):

$$\frac{B}{{B}_{ref}} = {( \frac{w}{{w}_{ref}} )}^{\frac{n + 1}{n}} \times {( \frac{{h}_{ref}}{h} )}^{2} , \qquad ( 5 )$$

the first average leg height similarity relationship is represented by Equation (6):

$$\frac{{H}_{1}}{{H}_{1 ref}} = \sqrt{{( \frac{w}{{w}_{ref}} )}^{\frac{n + 1}{n}} \times \frac{{w}_{ref} + 2 \times {B}_{ref}}{w + 2 \times B}} \qquad ( 6 )$$

the second average leg height similarity relationship is represented by Equation (7):

$$\frac{{H}_{2}}{{H}_{2 ref}} = \sqrt{{( \frac{w}{{w}_{ref}} )}^{\frac{n + 1}{n}} \times \frac{{w}_{2 ref}}{{w}_{2}}} \qquad ( 7 )$$

In Equations (5), (6), and (7), B denotes the overflow trough wall thickness of the designed overflow brick, B_{ref} denotes the overflow trough wall thickness of the reference overflow brick, H₁ denotes a first average leg height of the designed overflow brick, H₂ denotes a second average leg height of the designed overflow brick, W₂ denotes a leg width corresponding to the second average leg height H₂ of the designed overflow brick, W_{2ref} denotes a leg width corresponding to the second average leg height Href of the reference overflow brick, n denotes a creep stress exponent of the designed overflow brick and the reference overflow brick, W denotes an overflow surface width of the designed overflow brick, W_{ref} denotes the overflow surface width of the reference overflow brick, h_{ref} denotes the overflow trough depth at the inlet cross-section of the reference overflow brick, and h denotes an overflow trough depth at the inlet cross-section of the designed overflow brick.

In some embodiments, the overflow trough wall thickness B of the designed overflow brick is determine based on Equation (8):

$$B = {( \frac{w}{{w}_{ref}} )}^{\frac{n + 1}{n}} \times {( \frac{{h}_{ref}}{h} )}^{2} \times {B}_{ref} . \qquad ( 8 )$$

In some embodiments, the system of equations regarding the first height h₁ and the second height h₂ at the inlet cross-section of the designed overflow brick includes:
Equation (9) regarding the proximal leg height H_{I} of the designed overflow brick:

$${H}_{I} = {h}_{1} - [ \frac{w + 2 \times B}{2 \times tan \frac{θ}{2}} - ( \frac{R}{sin \frac{θ}{2}} - R ) - {h}_{2} ] , \qquad ( 9 )$$

Equation (10) regarding the distal leg height H₀ of the designed overflow brick:

$${H}_{0} = {H}_{I} + h - W \times tan ϕ , \qquad ( 10 )$$

Equation (11) regarding the first average leg height H₁ of the designed overflow brick:

$${H}_{1} = \frac{{H}_{I} + {H}_{0}}{2} , \qquad ( 11 )$$

Equation (12) regarding the second average leg height H₂ of the designed overflow brick:

$${H}_{2} = {H}_{1} + ( {h}_{1} - {H}_{I} ) , \qquad ( 12 )$$

Equation (13) regarding the leg width w₂ corresponding to the second average leg height H₂ of the designed overflow brick:

$${w}_{2} = ( w + 2 \times B ) - \frac{1}{{H}_{2}} \times {( {H}_{2} - {H}_{1} )}^{2} \times tan \frac{θ}{2} , \qquad ( 13 )$$

Equation (14) regarding the first average leg height H₁ of the designed overflow brick, which is determined based on the first average leg height similarity relationship:

$${H}_{1} = {H}_{1 ref} \times \sqrt{{( \frac{w}{{w}_{ref}} )}^{\frac{n + 1}{n}} \times \frac{{w}_{ref} + 2 \times {B}_{ref}}{w + 2 \times B}} , \qquad ( 14 )$$

Equation (15) regarding the second average leg height H₂ of the designed overflow brick, which is determined based on the second average leg height similarity relationship:

$${H}_{2} = {H}_{2 ref} \times \sqrt{{( \frac{w}{{w}_{ref}} )}^{\frac{n + 1}{n}} \times \frac{{w}_{2 ref}}{{w}_{2}}} . \qquad ( 15 )$$

In Equation (9), R denotes an overflow brick tip chamfer radius of the designed overflow brick, θ denotes an overflow inclined surface angle of the designed overflow brick, and φ denotes an overflow weir inclination angle of the designed overflow brick.

In some embodiments, the strength parameters include an equivalent thickness, an equivalent height base value, and an equivalent height, and the determining strength parameters of the reference overflow brick and the designed overflow brick includes:
determining an equivalent thickness of the reference overflow brick and an equivalent thickness of the designed overflow brick based on Equation (16) and Equation (17), respectively:

$$b = ( w + 2 \times B ) - 2 \times ( {h}_{1} - {H}_{I} ) \times tan \frac{θ}{2} , \qquad ( 16 )$$

$${b}_{ref} = ( {w}_{ref} + 2 \times {B}_{ref} ) - 2 \times ( {h}_{1 ref} - {H}_{Iref} ) \times tan \frac{{θ}_{ref}}{2} , \qquad ( 17 )$$

where, b_{ref} denotes the equivalent thickness of the reference overflow brick, and b denotes the equivalent thickness of the designed overflow brick;
determining an equivalent height base value of the reference overflow brick and an equivalent height base value of the designed overflow brick based on Equation (18) and Equation (19), respectively:

$${d}^{′} = \frac{1}{2 \times b} \times ( w + 2 B ) \times ( h + {h}_{1} + {h}_{2} + {H}_{0} ) , \qquad ( 18 )$$

$${d}_{ref}^{′} = \frac{1}{2 \times {b}_{ref}} \times ( {w}_{ref} + 2 {B}_{ref} ) \times ( {h}_{ref} + + {h}_{2 ref} + {H}_{0 ref} ) , \qquad ( 19 )$$

where, d′_{ref} denotes the equivalent height base value of the reference overflow brick, and d′ denotes the equivalent height base value of the designed overflow brick; and
determining an equivalent height d of the designed overflow brick and an equivalent height d_{ref} of the reference overflow brick based on Equation (20) and Equation (21), respectively:

$$d = {d}^{′} \times ( - 1.718936 \times 1 {0}^{- 5} \times ( b \times {d}^{′} \times W \times {ρ}_{y} ) + 1.169 \; 457 ) , \qquad ( 20 )$$

$${d}_{ref} = {d}_{ref}^{′} \times ( - 1.718936 \times 1 {0}^{- 5} \times ( {b}_{ref} \times {d}_{ref}^{′} \times {W}_{ref} \times {ρ}_{y} ) + 1.169 \; 457 , \qquad ( 21 )$$

where, b denotes the equivalent thickness of the designed overflow brick, b_{ref} denotes the equivalent thickness of the reference overflow brick, d′ denotes the equivalent height base value of the designed overflow brick, d′_{ref} denotes the equivalent height base value of the reference overflow brick, d denotes the equivalent height of the designed overflow brick, d_{ref} denotes the equivalent height of the reference overflow brick, H_{Oref} denotes the distal leg height of the reference overflow brick, and ρ_{y} denotes a material density of the designed overflow brick and the reference overflow brick.

In some embodiments, the support surface length l of the designed overflow brick is determined based on Equation (22):

$$l = {( \frac{d}{{d}_{ref}} )}^{2} \times \frac{{w}_{ref}}{w} \times {l}_{ref} , \qquad ( 22 )$$

where, l_{ref} denotes a support surface length of the reference overflow brick.

In some embodiments, the maximum cylinder pressure Pₘₐₓ of the clamp cylinder for the designed overflow brick is determined based on Equation (23):

$${P}_{max} = 1.251 \times W \times b \times d \times {p}_{y} , \qquad ( 23 )$$

where, d denotes the equivalent height of the designed overflow brick, b denotes the equivalent thickness of the designed overflow brick, and W denotes the overflow surface width of the designed overflow brick.

In some embodiments, the method further includes: fabricating a green body based on production parameters via an automated production unit, sintering the green body to form the designed overflow brick, and installing the designed overflow brick into a forming apparatus. The production parameters include the design parameters of the designed overflow brick, the overflow trough wall thickness B of the designed overflow brick, the first height h₁ and the second height h₂ at the inlet cross-section of the designed overflow brick, the support surface length l of the designed overflow brick, and the maximum cylinder pressure Pₘₐₓ of the clamp cylinder.

In some embodiments, the method further includes: setting a cylinder pressure of the clamp cylinder to an initial working pressure and monitoring a vibration state of the designed overflow brick via a vibration sensor; and, in response to the vibration sensor detecting a vibration signal characterizing abnormal creep deformation, increasing the cylinder pressure of the clamp cylinder from the initial working pressure to an enhanced constraint pressure. The initial working pressure is less than the enhanced constraint pressure, and the enhanced constraint pressure is less than the maximum cylinder pressure Pₘₐₓ of the clamp cylinder.

One or more embodiments of the present disclosure provide a high-strength low-creep overflow brick, which is designed and manufactured based on the method for designing the high-strength low-creep overflow brick provided in the present disclosure.

The beneficial effects of one or more embodiments of the present disclosure include at least the following. The method for designing a high-strength low-creep overflow brick provided in the present disclosure uses a mature overflow brick for glass substrate manufacturing as a design reference, obtaining the design parameters of the reference overflow brick, the design parameters of the designed overflow brick, and the creep stress exponent. The method further establishes similarity relationships between the reference overflow brick and the designed overflow brick, and constructs a system of equations regarding the first height and the second height at the inlet cross-section of the designed overflow brick. By applying the similarity relationships and the known parameters, the equivalent thickness, the equivalent height base value, and the equivalent height of each of the reference overflow brick and the designed overflow brick are obtained. Subsequently, the support surface length of the designed overflow brick and the maximum cylinder pressure of the clamp cylinder are determined based on the known parameters, thereby completing the design of the high-strength low-creep overflow brick. The method enables effective design optimization of the structural strength of the overflow brick, the clamping cylinder process, and long-term creep behavior, thereby ensuring the stability of the formed sheet quality.

### BRIEF DESCRIPTION OF THE DRAWINGS

The present disclosure will be further illustrated by way of exemplary embodiments, which will be described in detail through the accompanying drawings. These embodiments are not limiting, and in these embodiments, the same numerals represent the same structures.

FIG. 1 is a schematic structural diagram illustrating an overflow system according to some embodiments of the present disclosure.

FIG. 2 is a schematic diagram illustrating an overflow down-draw glass sheet according to some embodiments of the present disclosure.

FIG. 3 is a schematic diagram illustrating a force-bearing support structure of an overflow brick according to some embodiments of the present disclosure.

FIG. 4 is a schematic diagram illustrating a force-bearing structure of a clamp cylinder for an overflow brick according to some embodiments of the present disclosure.

FIG. 5 is a schematic diagram illustrating structural details of an overflow brick according to some embodiments of the present disclosure.

FIGS. 6A-6B are schematic diagrams illustrating a volumetric flow distribution and a differential volumetric flow distribution of an overflow brick according to some embodiments of the present disclosure, wherein, FIG. 6A shows the volumetric flow distribution, and FIG. 6B shows the differential volumetric flow distribution.

FIGS. 7A-7B are schematic diagrams illustrating a structure of an overflow brick before creep and a structure of the overflow brick after creep according to some embodiments of the present disclosure, wherein, FIG. 7A shows the structure before creep, and FIG. 7B shows the structure after creep.

Numeral references in the drawings: 1 denotes an overflow brick, 2 denotes an overflow trough, 3 denotes a molten glass feeding device, 4 denotes an overflow brick root, 5 denotes a flow guide plate, 6 denotes a formed glass substrate, 7 denotes a glass substrate down-draw direction, W_{G} denotes a specification width of the glass substrate, W_{Y} denotes a drawing width for the glass substrate, B denotes an overflow trough wall thickness of a designed overflow brick, W denotes an overflow surface width of the designed overflow brick, w denotes an overflow trough width of the designed overflow brick, h denotes an overflow trough depth at an inlet cross-section of the designed overflow brick, R denotes an overflow brick tip chamfer radius of the designed overflow brick, φ denotes an overflow weir inclination angle of the designed overflow brick, θ denotes an overflow inclined surface angle of the designed overflow brick, h₁ denotes a first height at the inlet cross-section of the designed overflow brick, h₂ denotes a second height at the inlet cross-section of the designed overflow brick, H_{O} denotes a distal leg height of the designed overflow brick, H_{I} denotes a proximal leg height of the designed overflow brick, L denotes a total length of the overflow brick, and I denotes a support surface length of the designed overflow brick.

### DETAILED DESCRIPTION

To make the objectives, technical solutions, and advantages of the embodiments of the present disclosure clearer, the technical solutions in the embodiments of the present disclosure will be described in detail below with reference to the accompanying drawings in the embodiments of the present disclosure. Obviously, the described embodiments are part, but not all, of the embodiments of the present disclosure.

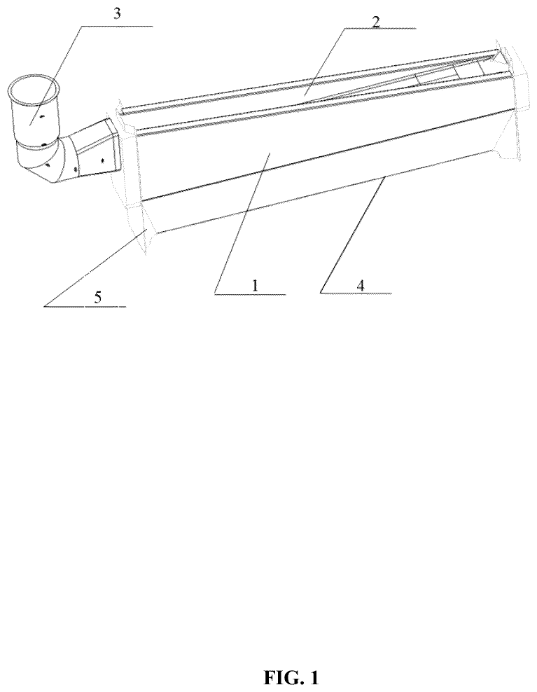

Referring to FIG. 1, an overflow system is composed of an overflow brick 1 connected to a molten glass feeding device 3. An overflow trough 2 is formed inside the overflow brick 1, and the bottom of the overflow brick 1 is an overflow brick root 4. During the manufacture of a glass substrate via a fusion overflow process, in a forming stage, molten glass melted in a glass melting furnace is supplied to the molten glass feeding device 3 of a fusion overflow forming apparatus. The molten glass then flows along the overflow trough 2, overflows from two sides of the overflow brick 1, and forms the glass substrate below the overflow brick root 4.

As the molten glass advances from a proximal end to a distal end of the overflow trough, it is driven by a body force and a pressure along a flow direction, overcomes laminar viscous resistance to move forward, and flows downward over an overflow weir. The hydrodynamic equations based on this principle integrate the effects of the aforementioned forces and form the foundation for the design of the overflow trough. On a vertical overflow surface, where the body force and the pressure are sufficiently large and the viscosity is relatively low, the influence of transverse surface tension is minimal, resulting in almost no transverse contraction. On an inclined surface, the components of the body force and the pressure along the inclined surface decrease significantly, and the viscosity gradually increases, causing the effect of transverse surface tension to become prominent and leading to noticeable transverse contraction. Therefore, a platinum flow guide plate 5 is provided at each of the proximal end and the distal end of the inclined surface of the overflow brick to partially counteract the transverse contraction of the glass.

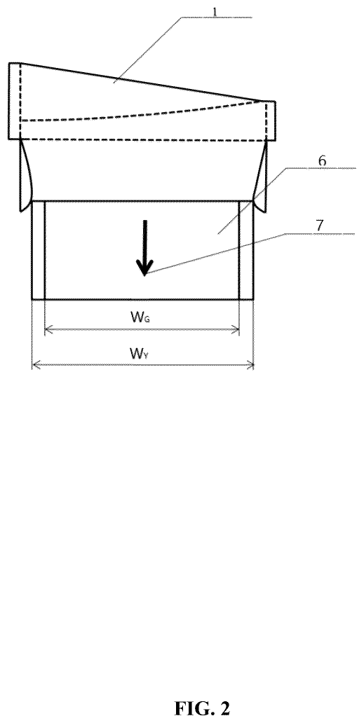

Referring to FIG. 2, the drawing process serves as the foundation for forming the glass substrate. During a down-draw forming process of the glass substrate, a formed glass substrate 6 moves downward along a down-draw direction 7 of the glass substrate. In FIG. 2, W_{G} denotes a specification width of the glass substrate, and W_{Y} denotes a drawing width for the glass substrate.

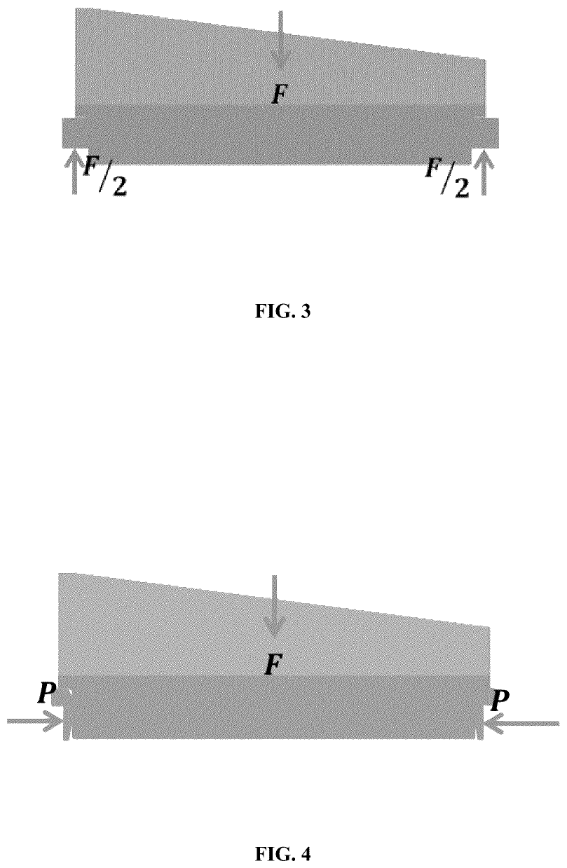

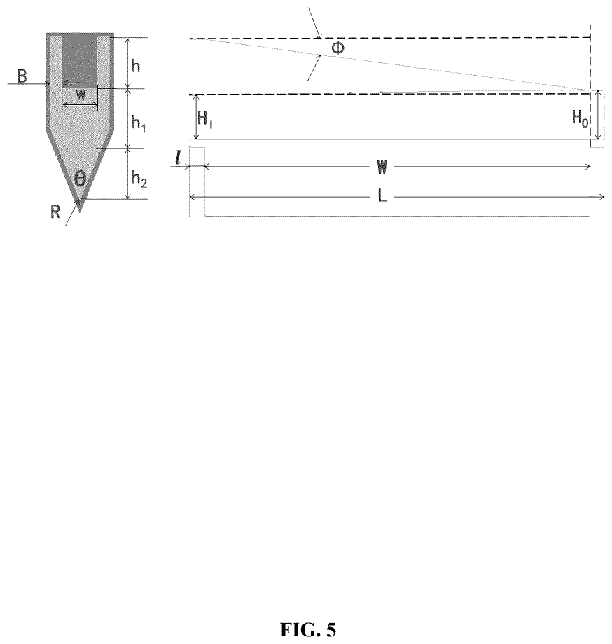

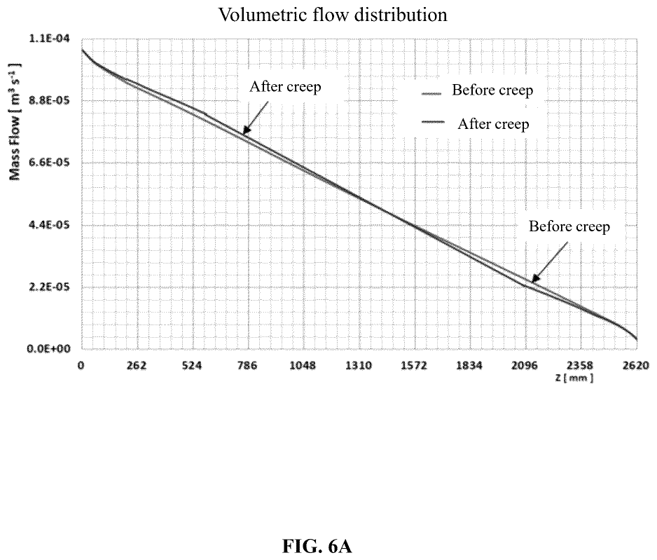

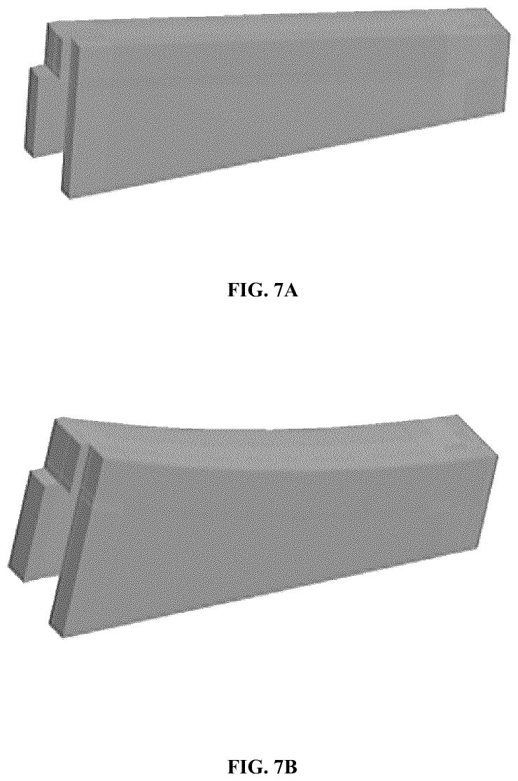

Referring to FIG. 3, a balance relationship must be established between a force on a support portion of the overflow brick and a gravity F (the gravity of the overflow brick and the glass) to ensure support strength. Referring to FIG. 4, a cylinder pressure P of a clamp cylinder on the overflow brick is required to resist the deformation of the overflow brick caused by the gravity F (the gravity of the overflow brick and the glass) as much as possible. Referring to FIGS. 5 and 7, long-term creep of the overflow brick causes downward bending, leading to a variation in the glass substrate where a middle portion of the overflow brick tends to thicken and edges of the overflow brick tend to thin, significantly affecting the stability of the drawing process. During the design of the overflow brick, various complex factors affecting a thickness distribution of the glass substrate must be considered, and production margins should be increased in the design to ensure that the thermal stability and stress of the overflow brick remain within a safe range of an ultimate flexural strength. The bending stress of the overflow brick includes self-weight stress, glass liquid gravity, pulling feedback stress, and clamp cylinder anti-bending stress, in which the pulling feedback stress is negligible. The average operating environment temperature is approximately 1207° C. A creep rate in an actual muffle furnace environment must be calculated to accurately estimate a creep amount of the overflow brick. Meanwhile, to facilitate accurate calculation, a series of creep rates under different environmental temperatures, external stresses, and holding times are measured. The impact of creep in the overflow brick on the formed thickness may be verified through forming overflow simulation or overflow brick design flow analysis. The creep in the overflow brick leads to a trend where a distal end and a proximal end of the formed glass substrate become thinner and the middle portion becomes thicker. Therefore, the structural design of the overflow brick must first consider flexural strength. At the same time, an appropriate cylinder pressure of the clamp cylinder is the basis for effectively controlling the creep amount. Additionally, a creep stress exponent n of the overflow brick is considered during the structural design of the overflow brick, ensuring that the strength and creep resistance of the designed overflow brick are higher than those of the reference overflow brick, and the creep amount of the overflow brick is controlled through a cylinder pressure process.

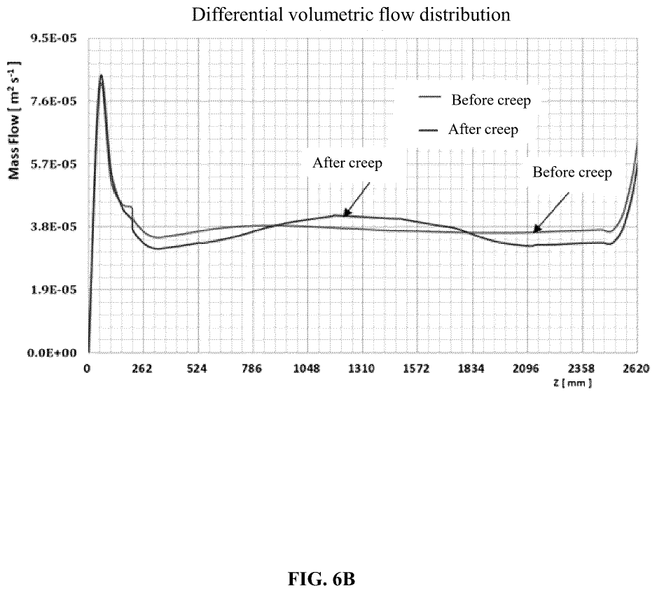

Referring to FIGS. 6A-6B, which are a set of schematic diagrams illustrating a volumetric flow distribution and a differential volumetric flow distribution of an overflow brick according to some embodiments of the present disclosure. FIG. 6A shows the volumetric flow distribution. Before creep occurs, the flow rate of the molten glass decreases approximately uniformly from the proximal end to the distal end of the formed glass substrate (approximating a straight downward-sloping line). After creep occurs, the flow rate decrease of the molten glass at the proximal end and the distal end slows down, resulting in an upward-curving trend of the curve, indicating that the amount of glass flowing out is reduced compared to before creep. Conversely, the flow rate decrease in the middle portion accelerates, indicated by a steeper downward slope of the curve, signifying an increased amount of glass flowing out compared to before creep. FIG. 6B shows the differential volumetric flow distribution, which is the derivative of the curve in FIG. 6A. The magnitude of the differential volumetric flow is proportional to the thickness distribution of the glass substrate. It can be observed that after creep, the glass substrate tends to become thicker in the middle portion and thinner at the distal end and the proximal end. The aforementioned changes severely affect the forming quality of the glass substrate and the stability of the drawing process.

Some embodiments of the present disclosure provide a method for designing a high-strength low-creep overflow brick. The method includes following operations.

A mature overflow brick for manufacturing a glass substrate is selected as a reference overflow brick, design parameters of the reference overflow brick are obtained, and design parameters of a designed overflow brick are determined.

In some embodiments, obtaining the design parameters of the reference overflow brick includes: obtaining an overflow trough width W_{ref} of the reference overflow brick, an overflow trough height h_{ref} of the reference overflow brick, an overflow trough wall thickness B_{ref} of the reference overflow brick; a first height h_{1ref} at an inlet cross-section of the reference overflow brick, a second height h_{2ref} at the inlet cross-section of the reference overflow brick, an overflow weir inclination angle φ_{ref} of the reference overflow brick, an overflow inclined surface angle θ_{ref} of the reference overflow brick, an overflow brick tip chamfer radius R_{ref} of the reference overflow brick, an overflow surface width W_{ref} of the reference overflow brick, and a support surface length l_{ref} of the reference overflow brick.

In some embodiments, determining the design parameters of the designed overflow brick includes: determining an overflow trough width w of the designed overflow brick and an overflow trough depth h of the designed overflow brick; an overflow weir inclination angle φ of the designed overflow brick, an overflow inclined surface angle θ of the designed overflow brick, an overflow brick tip chamfer radius R of the designed overflow brick, an overflow surface width W of the designed overflow brick, and a creep stress exponent n of the designed overflow brick and the reference overflow brick.

An overflow trough wall thickness similarity relationship, a first average leg height similarity relationship, and a second average leg height similarity relationship between the reference overflow brick and the designed overflow brick are established based on the design parameters of the reference overflow brick, the design parameters of the designed overflow brick, and the creep stress exponent n of the designed overflow brick and the reference overflow brick, to determine the overflow trough wall thickness of the designed overflow brick, and the first height and the second height at the inlet cross-section of the designed overflow brick.

Meanwhile, a support surface length similarity relationship between the reference overflow brick and the designed overflow brick is established. Based on parameters including the design parameters of the reference overflow brick, a proximal leg height H_{Iref} and a distal leg height H_{Oref} of the reference overflow brick, the design parameters of the designed overflow brick, the overflow trough wall thickness B, and the first height h₁ and the second height h₂ at the inlet cross-section of the designed overflow brick, strength parameters of the reference overflow brick and the designed overflow brick are determined.

In some embodiments, the strength parameters include an equivalent thickness, an equivalent height base value, and an equivalent height. Furthermore, a support surface length of the designed overflow brick and a maximum cylinder pressure Pₘₐₓ of the clamp cylinder without bending of the overflow brick are determined through calculation. A cylinder pressure process is established, thereby completing the design of the structural profile of the high-strength low-creep overflow brick.

In some embodiments, the overflow trough wall thickness similarity relationship between the reference overflow brick and the designed overflow brick may be expressed by Equation (5):

$$\frac{B}{{B}_{ref}} = {( \frac{w}{{w}_{ref}} )}^{\frac{n + 1}{n}} \times {( \frac{{h}_{ref}}{h} )}^{2} . \qquad ( 5 )$$

In Equation (5), B and B_{ref} denote the overflow trough wall thickness of the designed overflow brick and the overflow trough wall thickness of the reference overflow brick, respectively; W and W_{ref} denote the overflow surface width of the designed overflow brick and the overflow surface width of the reference overflow brick, respectively; h and h_{ref} denote the overflow trough depth at the inlet cross-section of the designed overflow brick and an overflow trough depth at the inlet cross-section of the reference overflow brick, respectively; and n denotes the creep stress exponent of the designed overflow brick and the reference overflow brick.

In some embodiments, the first average leg height similarity relationship between the reference overflow brick and the designed overflow brick may be expressed by Equation (6):

$$\frac{{H}_{1}}{{H}_{1 ref}} = {( \frac{w}{{w}_{ref}} )}^{\frac{n + 1}{n}} \times \frac{{w}_{ref} + 2 \times {B}_{ref}}{w + 2 \times B} . \qquad ( 6 )$$

In Equation (6), H₁ and H_{1ref} denote a first average leg height of the designed overflow brick and a first average leg height of the reference overflow brick, respectively; w and W_{ref} denote the overflow trough width of the designed overflow brick and the overflow trough width of the reference overflow brick, respectively; w+2×B denotes a leg width corresponding to the first average leg height H₁ of the designed overflow brick, i.e., a thickness of the designed overflow brick, formed by adding the overflow trough wall thickness of the designed overflow brick to the overflow trough width of the designed overflow brick.

In some embodiments, the second average leg height similarity relationship is represented by Equation (7):

$$\frac{{H}_{2}}{{H}_{2 ref}} = \sqrt{{( \frac{W}{{W}_{ref}} )}^{\frac{n + 1}{n}} \times \frac{{W}_{2 ref}}{{W}_{2}}} . \qquad ( 7 )$$

In Equation (7), H₂ and H_{2ref} denote a second average leg height of the designed overflow brick a second average leg height of the reference overflow brick, respectively, W₂ denotes a leg width corresponding to the second average leg height H₂ of the designed overflow brick, and W_{2ref} denotes a leg width corresponding to the second average leg height H_{2ref} of the reference overflow brick.

In some embodiments, the first average leg height of the designed overflow brick and the first average leg height of the reference overflow brick may be determined based on Equation (11) and Equation (3), respectively:

$${H}_{1} = \frac{{H}_{I} + {H}_{O}}{2} , \qquad ( 11 )$$

$${H}_{1 ref} = \frac{{H}_{Iref} + {H}_{Oref}}{2} , \qquad ( 3 )$$

wherein, H_{I} and H_{Iref} denote a proximal leg height of the designed overflow brick and a proximal leg height of the reference overflow brick, respectively, H_{O} and H_{Oref} denote a distal leg height of the designed overflow brick and a distal leg height of the reference overflow brick, respectively.

In some embodiments, the second average leg height of the designed overflow brick and the second average leg height of the reference overflow brick may be determined based on Equation (12) and Equation (4), respectively:

$${H}_{2} = {H}_{1} + ( {h}_{1} - {H}_{I} ) , \qquad ( 12 )$$

$${H}_{2 ref} = {H}_{1 ref} + ( {h}_{1 ref} - {H}_{Iref} ) , \qquad ( 4 )$$

wherein, h₁ and h_{ref} denote the first height at the inlet cross-section of the designed overflow brick and the first height at the inlet cross-section of the reference overflow brick, respectively.

In some embodiments, the proximal leg height of the designed overflow brick and the proximal leg height of the reference overflow brick may be determined based on Equation (9) and Equation (1), respectively:

$${H}_{I} = {h}_{1} - [ \frac{w + 2 \times B}{2 \times tan} - ( \frac{R}{sin \frac{θ}{2}} - R ) - {h}_{2} ] , \qquad ( 9 )$$

$${H}_{Iref} = {h}_{1 ref} - [ \frac{{w}_{ref} + 2 \times {B}_{ref}}{2 \times tan \frac{{θ}_{ref}}{2}} - ( \frac{{r}_{ref}}{sin \frac{{θ}_{ref}}{2}} - {R}_{ref} ) - {h}_{2 ref} ] , \qquad ( 1 )$$

where h₂ and h_{2ref} denote the second height at the inlet cross-section of the designed overflow brick and the second height at the inlet cross-section of the reference overflow brick, respectively; θ and θ_{ref} denote the overflow inclined surface angle of the designed overflow brick and the overflow inclined surface angle of the reference overflow brick, respectively; R and R_{ref} denote the overflow brick tip chamfer radius of the designed overflow brick and an overflow brick tip chamfer radius of the reference overflow brick, respectively.

In some embodiments, the distal leg height of the designed overflow brick and the distal leg height of the reference overflow brick may be determined based on Equation (10) and Equation (2), respectively:

$${H}_{O} - {H}_{I} + h - W \times tan ϕ , \qquad ( 10 )$$

$${H}_{Oref} = {H}_{Iref} + {h}_{ref} - {W}_{ref} \times tan {ϕ}_{ref} , \qquad ( 2 )$$

where h and h_{ref} denote the overflow trough depth of the designed overflow brick and the overflow trough depth of the reference overflow brick, respectively; φ and φ_{ref} denote the overflow weir inclination angle of the designed overflow brick and the overflow weir inclination angle of the reference overflow brick, respectively.

In some embodiments, the leg width w₂ corresponding to the second average leg height H₂ of the designed overflow brick, and the leg width w_{2ref} corresponding to the second average leg height H_{2ref} of the reference overflow brick, may be determined based on Equation (13) and Equation (24), respectively:

$${w}_{2} = ( w + 2 \times B ) - \frac{1}{{H}_{2}} \times {( {H}_{2} - {H}_{1} )}^{2} \times tan \frac{θ}{2} , \qquad ( 13 )$$

$${w}_{2 ref} = ( {w}_{ref} + 2 \times {B}_{ref} ) - \frac{1}{{H}_{2 ref}} \times {( {H}_{2 ref} - {H}_{1 ref} )}^{2} \times tan \frac{{θ}_{ref}}{2} . \qquad ( 24 )$$

In some embodiments, the support surface length similarity relationship between the reference overflow brick and the designed overflow brick may be expressed using Equation (25):

$$\frac{l}{{l}_{ref}} = {( \frac{d}{{d}_{ref}} )}^{2} \times \frac{{w}_{ref}}{w} , \qquad ( 25 )$$

where l and l_{ref} denote the support surface length of the designed overflow brick and the support surface length of the reference overflow brick, respectively; d and d_{ref} denote an equivalent height of the designed overflow brick and an equivalent height of the reference overflow brick, respectively.

In some embodiments, the equivalent height of the designed overflow brick and the equivalent height of the reference overflow brick may be determined based on Equation (20) and Equation (21), respectively:

$$d = {d}^{′} \times ( - 1.718936 \times {10}^{- 5} \times ( b \times {d}^{′} \times W \times {ρ}_{y} ) + 1.169457 ) , \qquad ( 20 )$$

$${d}_{ref} = {d}_{ref}^{′} \times ( - 1.718936 \times {10}^{- 5} \times ( {b}_{ref} \times {d}_{ref}^{′} \times {W}_{ref} \times {ρ}_{y} ) + 1.169457 ) , \qquad ( 21 )$$

where b and b_{ref} denote an equivalent thickness of the designed overflow brick and an equivalent thickness of the reference overflow brick, respectively; d′ and d′_{ref} denote an equivalent height base value of the designed overflow brick and an equivalent height base value of the reference overflow brick, respectively; ρ_{y} denotes a material density of the reference overflow brick and the designed overflow brick.

In some embodiments, the equivalent height base value of the designed overflow brick and the equivalent height base value of the reference overflow brick may be determined based on Equation (18) and Equation (19), respectively:

$${d}^{′} = \frac{1}{2 \times b} \times ( w + 2 B ) \times ( h + {h}_{1} + {h}_{2} + {H}_{O} ) , \qquad ( 18 )$$

$${d}_{ref}^{′} = \frac{1}{2 \times {b}_{ref}} \times ( {w}_{ref} + 2 {B}_{ref} ) \times ( {h}_{ref} + {h}_{1 ref} + {h}_{2 ref} + {H}_{Oref} ) . \qquad ( 19 )$$

In some embodiments, the equivalent thickness of the designed overflow brick and the equivalent thickness of the reference overflow brick may be determined based on Equation (16) and Equation (17), respectively:

$$b = ( w + 2 \times B ) - 2 \times ( {h}_{1} - {H}_{I} ) \times tan \frac{θ}{2} , \qquad ( 16 )$$

$${b}_{ref} = ( {w}_{ref} + 2 \times {B}_{ref} ) - 2 \times ( {h}_{1 ref} - {H}_{Iref} ) \times tan \frac{{θ}_{ref}}{2} . \qquad ( 17 )$$

In some embodiments, the maximum cylinder pressure of the clamp cylinder without bending for the designed overflow brick may be determined based on Equation (23):

$${P}_{max} = 1.251 \times W \times b \times d \times {ρ}_{y} , \qquad ( 23 )$$

where Pₘₐₓ denotes the maximum cylinder pressure of the clamp cylinder without bending for the designed overflow brick, with unit in kg; ρ_{y} denotes the material density of the designed overflow brick.

In some embodiments, during an initial use phase of the overflow brick, before creep occurs, the deformation amount of the overflow brick is on the order of micrometers. An initial pressure of the clamp cylinder before drawing (e.g., during heating and wetting) is approximately 0.85×Pₘₐₓ. After drawing, the cylinder pressure of the clamp cylinder is recommended to be within the range of (0.90 to 0.95)×Pₘₐₓ. As a creep amount of the overflow brick continuously increases, a clamping force from the clamp cylinder should not be increased beyond a clamping force corresponding to the maximum cylinder pressure Pₘₐₓ.

In some embodiments, the method for designing the high-strength low-creep overflow brick disclosed in the present disclosure further includes: setting the cylinder pressure of the clamp cylinder to an initial working pressure and monitoring a vibration state of the designed overflow brick via a vibration sensor; and, in response to the vibration sensor detecting a vibration signal characterizing abnormal creep deformation, increasing the cylinder pressure of the clamp cylinder from the initial working pressure to an enhanced constraint pressure. The initial working pressure is less than the enhanced constraint pressure, and the enhanced constraint pressure is less than the maximum cylinder pressure Pₘₐₓ of the clamp cylinder.

The clamp cylinder is an actuator configured to apply a controllable compressive force to a side surface of the designed overflow brick. The purpose of using the clamp cylinder is to counteract a downward bending stress caused by a self-weight of the overflow brick and a weight of the molten glass, and to actively suppress the creep deformation of the overflow brick during long-term high-temperature service.

In some embodiments, a pair of clamp cylinders are installed on two sides of the designed overflow brick to ensure the applied clamping forces are balanced, thereby avoiding the generation of additional torsional stress.

In some embodiments, the clamp cylinders may be pneumatic cylinders or hydraulic cylinders. Precise control of the cylinder pressure for the clamp cylinders may be implemented based on a Programmable Logic Controller (PLC).

The initial working pressure refers to an initial pressure applied by the clamp cylinders during the initial use phase of the designed overflow brick (e.g., during the heating and wetting stages before the drawing process). The initial working pressure provides a basic structural constraint and ensures a safe process start-up. For example, the initial working pressure may be set to about 85% (e.g., within the range of 80% to 90%) of the maximum cylinder pressure Pₘₐₓ.

The vibration sensor is a device configured to detect mechanical vibrations. In some embodiments, the vibration sensor may monitor the vibration of the designed overflow brick in real-time to indirectly indicate whether creep deformation or other structural abnormalities occur. In some embodiments, to improve monitoring sensitivity and accuracy, the vibration sensor may be installed at a key structural portion of the designed overflow brick, such as the overflow brick root or a support region (i.e., a region where the designed overflow brick interfaces with and is supported by the forming apparatus) of the designed overflow brick.

The vibration state of the designed overflow brick reflects its structural stability under high temperature and gravity. Under normal operating conditions, the vibration signal of the designed overflow brick exhibits stable, low-amplitude characteristics. When creep initiates or intensifies, internal stress of the overflow brick redistributes, potentially causing changes in a natural frequency of the overflow brick or generating irregular micro-vibrations, which may be detected by the vibration sensor.

The vibration signal refers to data obtained after a raw signal collected by the vibration sensor undergoes signal processing and analysis. In some embodiments, the raw signal collected by the vibration sensor is a time-domain waveform signal. To extract effective features, a Manufacturing Execution System (MES) may first process the raw signal, for example, by converting the raw signal into a frequency-domain signal (spectrum) using a Fast Fourier Transform (FFT).

The vibration signal characterizing abnormal creep deformation is a judgment basis for triggering the pressure increase. In some embodiments, the vibration signal characterizing abnormal creep deformation may manifest as one or more of the following: an amplitude of the vibration signal continuously increases over a period and exceeds a preset alarm threshold; or a frequency of the vibration signal changes abruptly, e.g., showing a frequency component higher than a normal operating range. These phenomena indicate that the creep deformation of the designed overflow brick is intensifying.

In some embodiments, the alarm threshold and the normal operating range may be preset manually.

The enhanced constraint pressure refers to a preset pressure level to which the working pressure of the clamp cylinder is elevated after the system detects a vibration signal characterizing abnormal creep deformation. The enhanced constraint pressure provides a stronger structural constraint force to actively inhibit further development of the creep deformation in the designed overflow brick, thereby maintaining the geometric stability and forming accuracy of the designed overflow brick. The preset pressure level is a predetermined value, for example, a value set manually.

In some embodiments, to ensure effective constraint without exceeding the structural safety limits, the enhanced constraint pressure may be less than the maximum cylinder pressure Pₘₐₓ. For example, the enhanced constraint pressure may be set within a range of 90% to 95% of the maximum cylinder pressure Pₘₐₓ, thereby ensuring that the enhanced constraint pressure significantly improves the suppression of creep while maintaining a safety margin.

In some embodiments, increasing the working pressure of the clamp cylinder is not performed instantaneously but follows a predefined pressure ramp curve to achieve smooth and safe control. This process may include the following stages. During an initial process phase, such as before drawing, the MES sets and maintains the cylinder pressure of the clamp cylinder at the initial working pressure (e.g., 0.85×Pₘₐₓ), focusing on preheating and stabilizing the overflow brick. In a mid-process phase, such as after drawing, if the vibration sensor detects a vibration signal characterizing abnormal creep deformation, the MES initiates a pressure increase procedure, gradually raising the initial working pressure to the enhanced constraint pressure (e.g., 0.95×Pₘₐₓ). To avoid structural impact on the overflow brick from sudden pressure changes, this increase process may adopt a gradual adjustment strategy, such as incrementally raising the initial working pressure by a small amount at preset time intervals until a target value is reached. In a later process phase, if the monitored creep rate stabilizes, the MES may appropriately reduce the pressure from the enhanced constraint pressure back to a stable working pressure and enter a maintenance mode, where adjustments are only triggered when detecting anomalies again.

In some embodiments of the present disclosure, by introducing a dynamic pressure adjustment mechanism based on real-time monitoring, the clamping force (i.e., the cylinder pressure) may be dynamically optimized according to the actual state of the overflow brick. This approach ensures that the overflow brick remains in an optimal constrained state for resisting creep throughout its entire service life, avoiding potential damage from excessively high pressure or insufficient constraint from overly low pressure, thereby significantly extending the effective service life and maintenance cycle of the overflow brick.

In some embodiments, the method for designing the high-strength low-creep overflow brick includes the following operations.

The design parameters related to the reference overflow brick are obtained, including: the overflow trough width W_{ref} of the reference overflow brick, the overflow trough height h_{ref} of the reference overflow brick, the overflow trough wall thickness B_{ref} of the reference overflow brick, the first height hirer at the inlet cross-section of the reference overflow brick, the second height h_{2ref} at the inlet cross-section of the reference overflow brick, the overflow weir inclination angle φ_{ref} of the reference overflow brick, the overflow inclined surface angle θ_{ref} of the reference overflow brick, the overflow brick tip chamfer radius R_{ref} of the reference overflow brick, the overflow surface width W_{ref} of the reference overflow brick, and the support surface length I_{ref} of the reference overflow brick

The design parameters related to the designed overflow brick are obtained, including: the overflow trough width w of the designed overflow brick, the overflow trough depth h of the designed overflow brick, the overflow weir inclination angle φ of the designed overflow brick, the overflow inclined surface angle θ of the designed overflow brick, the overflow brick tip chamfer radius R of the designed overflow brick, and the overflow surface width W of the designed overflow brick

The overflow trough wall thickness of the designed overflow brick is determined based on Equation (8):

$$B = {( \frac{w}{{w}_{ref}} )}^{\frac{n + 1}{n}} \times {( \frac{{h}_{ref}}{h} )}^{2} \times {B}_{ref} . \qquad ( 8 )$$

In Equation (8), B and B_{ref} denote the overflow trough wall thickness of the designed overflow brick and the overflow trough wall thickness of the reference overflow brick, respectively; W and W_{ref} denote the overflow surface width of the designed overflow brick and the overflow surface width of the reference overflow brick, respectively; h and h_{ref} denote the overflow trough depth at the inlet cross-section of the designed overflow brick and the overflow trough depth at the inlet cross-section of the reference overflow brick, respectively; and n denotes the creep stress exponent of the designed overflow brick and the reference overflow brick.

In some embodiments, the creep stress exponent n of the overflow brick may be measured by conducting creep tests on the overflow brick, which specifically includes following operations.

For an overflow brick of a specific material, under a temperature T and an applied constant stress σ, a creep rate \acute{ε} is measured. The creep rate \acute{ε} conforms to the power-law representation, i.e., Equation (26):

$${ε}^{'} = \frac{d ε}{dt} = f ( σ , T ) = A \times {σ}^{n} \times exp ( - \frac{Q}{T} ) \approx \frac{DR}{t} = \frac{Bending Deflection}{Span ( Distance between Supports} / t . \qquad ( 26 )$$

In Equation (26), ε denotes a bending creep strain (unit: mm/mm), \acute{ε} denotes the creep rate (unit: mm/mm/hr), t denotes time, A denotes a material-dependent coefficient related to stress and temperature, n denotes the creep stress exponent of the overflow brick, Q=ER, where E denotes activation energy, and R denotes a gas constant, both of which may be determined experimentally. DR refers to a quantity related to bending deflection. (DR/t) represents an equivalent creep rate \acute{ε} calculated from the bending deflection. The creep rate is specified for a particular measurement temperature and applied stress.
the first height h₁ and the second height h₂ at the inlet cross-section of the designed overflow brick are determined through the following operations S11-S17.

In S11, the proximal leg height of the designed overflow brick and the proximal leg height of the reference overflow brick (where the values of h₁ and h₂ are currently unknown) may be determined based on Equation (9) and Equation (1), respectively:

$${H}_{I} = {h}_{1} - [ \frac{w + 2 \times B}{2 \times tan} - ( \frac{R}{sin \frac{θ}{2}} - R ) - {h}_{2} ] , \qquad ( 9 )$$

$${H}_{Iref} = {h}_{1 ref} - [ \frac{{w}_{ref} + 2 \times {B}_{ref}}{2 \times tan \frac{{θ}_{ref}}{2}} - ( \frac{{r}_{ref}}{sin \frac{{θ}_{ref}}{2}} - {R}_{ref} ) - {h}_{2 ref} ] , \qquad ( 1 )$$

where w and w_{ref} denote the overflow trough width of the designed overflow brick and the overflow trough width of the reference overflow brick, respectively; h₁ and h_{1ref} denote the first height at the inlet cross-section of the designed overflow brick and the first height at the inlet cross-section of the reference overflow brick, respectively; h₂ and h_{2ref} denote the second height at the inlet cross-section of the designed overflow brick and the second height at the inlet cross-section of the reference overflow brick, respectively; θ and θ_{ref} denote the overflow inclined surface angle of the designed overflow brick and the overflow inclined surface angle of the reference overflow brick, respectively; R and R_{ref} denote the overflow brick tip chamfer radius of the designed overflow brick and the overflow brick tip chamfer radius of the reference overflow brick, respectively.

In S12, the distal leg height of the designed overflow brick and the distal leg height of the reference overflow brick may be determined based on Equation (10) and Equation (2), respectively (where the values of h₁ and h₂ are currently unknown):

$${H}_{O} - {H}_{I} + h - W \times tan ϕ , \qquad ( 10 )$$

$${H}_{Oref} = {H}_{Iref} + {h}_{ref} - {W}_{ref} \times tan {ϕ}_{ref} , \qquad ( 2 )$$

where h and h_{ref} denote the overflow trough depth of the designed overflow brick and the overflow trough depth of the reference overflow brick, respectively; φ and φ_{ref} denote the overflow weir inclination angle of the designed overflow brick and the overflow weir inclination angle of the reference overflow brick, respectively.

In S13, the first average leg height of the designed overflow brick and the first average leg height of the reference overflow brick may be determined based on Equation (11) and Equation (3), respectively (where the values of h₁ and h₂ are currently unknown):

$${H}_{1} = \frac{{H}_{I} + {H}_{O}}{2} , \qquad ( 11 )$$

$${H}_{1 ref} = \frac{{H}_{Iref} + {H}_{Oref}}{2} . \qquad ( 3 )$$

In S14, the second average leg height of the designed overflow brick and the second average leg height of the reference overflow brick may be determined based on Equation (12) and Equation (4), respectively (where the values of h₁ and h₂ are currently unknown):

$${H}_{2} = {H}_{1} + ( {h}_{1} - {H}_{I} ) , \qquad ( 12 )$$

$${H}_{2 ref} = {H}_{1 ref} + ( {h}_{1 ref} - {H}_{Iref} ) . \qquad ( 4 )$$

In S15, the leg width corresponding to the second average leg height of the designed overflow brick and the leg width corresponding to the second average leg height of the reference overflow brick may be determined based on Equation (13) and Equation (24), respectively (where the values of h₁ and h₂ are currently unknown):

$${w}_{2} = ( w + 2 \times B ) - \frac{1}{{H}_{2}} \times {( {H}_{2} - {H}_{1} )}^{2} \times tan \frac{θ}{2} , \qquad ( 13 )$$

$${w}_{2 ref} = ( {w}_{ref} + 2 \times {B}_{ref} ) - \frac{1}{{H}_{2 ref}} \times {( {H}_{2 ref} - {H}_{1 ref} )}^{2} \times tan \frac{{θ}_{ref}}{2} . \qquad ( 24 )$$

In S16, the first average leg height of the designed overflow brick may be determined based on Equation (14) (where the values of h₁ and h₂ are currently unknown):

$${H}_{1} = {H}_{1 ref} \times \sqrt{{( \frac{W}{{W}_{ref}} )}^{\frac{n + 1}{n}} \times \frac{{w}_{ref} + 2 \times {B}_{ref}}{w + 2 \times B}} . \qquad ( 14 )$$

In S17, the second average leg height of the designed overflow brick may be determined based on Equation (15) (where the values of h₁ and h₂ are currently unknown):

$${H}_{2} = {H}_{2 ref} \times \sqrt{{( \frac{W}{{W}_{ref}} )}^{\frac{n + 1}{n}} \times \frac{{w}_{2 ref}}{{w}_{2}}} . \qquad ( 15 )$$

In S18, a system of equations consisting of the equations from operations S11-S77 may be established, and the system of equations may be solved to obtain the first height h₁ and the second height h₂ at the inlet cross-section of the designed overflow brick.

The method for designing the high-strength low-creep overflow brick further includes determining the support surface length l of the designed overflow brick, which may include the following operations S21-S24.

In S21, the equivalent thickness of the designed overflow brick and the equivalent thickness of the reference overflow brick may be determined based on Equation (16) and Equation (17), respectively:

$$b = ( w + 2 \times B ) - 2 \times ( {h}_{1} - {H}_{I} ) \times tan \frac{θ}{2} , \qquad ( 16 )$$

$${b}_{ref} = ( {w}_{ref} + 2 \times {B}_{ref} ) - 2 \times ( {h}_{1 ref} - {H}_{Iref} ) \times tan \frac{{θ}_{ref}}{2} . \qquad ( 17 )$$

In S22, the equivalent height base value of the designed overflow brick and the equivalent height base value of the reference overflow brick may be determined based on Equation (18) and Equation (19), respectively:

$${d}^{′} = \frac{1}{2 \times b} \times ( w + 2 B ) \times ( h + {h}_{1} + {h}_{2} + {H}_{O} ) , \qquad ( 18 )$$

$${{d}^{′}}_{ref} = \frac{1}{2 \times {b}_{ref}} \times ( {w}_{ref} + 2 {B}_{ref} ) \times ( {h}_{ref} + {h}_{1 ref} + {h}_{2 ref} + {H}_{Oref} ) . \qquad ( 19 )$$

In S23, the equivalent height of the designed overflow brick and the equivalent height of the reference overflow brick may be determined based on Equation (20) and Equation (21), respectively:

$$d = {d}^{′} \times ( - 1.718936 \times {10}^{- 5} \times ( b \times {d}^{′} \times W \times {ρ}_{y} ) + 1.169457 ) , \qquad ( 20 )$$

$${d}_{ref} = {{d}^{′}}_{ref} \times ( - 1.718936 \times {10}^{- 5} \times ( {b}_{ref} \times {{d}^{′}}_{ref} \times {W}_{ref} \times {ρ}_{y} ) + 1.169457 ) , \qquad ( 21 )$$

where b and b_{ref} denote the equivalent thickness of the designed overflow brick and the equivalent thickness of the reference overflow brick, respectively; d′ and d′_{ref} denote the equivalent height base value of the designed overflow brick and the equivalent height base value of the reference overflow brick, respectively; d and d_{ref} denote the equivalent height of the designed overflow brick and the equivalent height of the reference overflow brick, respectively; ρ_{y} denotes the material density of the reference overflow brick and the designed overflow brick.

In S24, the support surface length l of the designed overflow brick may be determined based on Equation (22):

$$l = {( \frac{d}{{d}_{ref}} )}^{2} \times \frac{{W}_{ref}}{W} \times {l}_{ref} . \qquad ( 22 )$$

The maximum cylinder pressure of the clamp cylinder without bending for the designed overflow brick may be determined based on Equation (23):

$${P}_{max} = 1.251 \times W \times b \times d \times {ρ}_{y} . \qquad ( 23 )$$

During the initial use phase of the overflow brick, before significant creep occurs, the deformation of the overflow brick is on the order of micrometers. The initial working pressure of the clamp cylinder before drawing (e.g., during heating and wetting) is approximately 0.85×Pₘₐₓ. After drawing, the cylinder pressure of the clamp cylinder is recommended to be within the range of (0.90 to 0.95)×Pₘₐₓ. As the creep amount of the overflow brick continuously increases, the clamping force from the clamp cylinder should not be increased beyond the clamping force corresponding to the maximum cylinder pressure Pₘₐₓ.

In some embodiments of the present disclosure, by using a mature overflow brick for glass substrate manufacturing as a design reference, the design parameters of the reference overflow brick, the design parameters of the target designed overflow brick, and the creep stress exponent are obtained. Similarity relationships between the reference overflow brick and the designed overflow brick are established, and a system of equations concerning the first and second heights at the inlet cross-section of the designed overflow brick is constructed. By applying the similarity relationships and known parameters, the equivalent thickness, the equivalent height base value, and the equivalent height of both the reference overflow brick and the designed overflow brick are obtained. Subsequently, the support surface length of the designed overflow brick and the maximum cylinder pressure of the clamp cylinder are determined based on the known parameters, thereby completing the design of the high-strength low-creep overflow brick. The method enables effective design optimization of the structural strength of the overflow brick, the clamp cylinder process, and long-term creep behavior, thereby ensuring the stability of the formed sheet quality.

In some embodiments, the method for designing the high-strength low-creep overflow brick disclosed in the present disclosure further includes: fabricating a green body based on production parameters via an automated production unit, sintering the green body to form the designed overflow brick, and installing the designed overflow brick into the forming apparatus. The production parameters include the design parameters of the designed overflow brick, the overflow trough wall thickness B of the designed overflow brick, the first height h₁ and the second height h₂ at the inlet cross-section of the designed overflow brick, the support surface length l of the designed overflow brick, and the maximum cylinder pressure Pₘₐₓ of the clamp cylinder.

In some embodiments, the MES receives and parses the production parameters to automatically generate a three-dimensional (3D) digital model. The automated production unit, based on the 3D digital model, automatically executes the entire workflow from raw material processing to finished product machining, ultimately producing the designed overflow brick.

The Manufacturing Execution System (MES) refers to an information management system responsible for coordinating and controlling the entire manufacturing process. In some embodiments, the MES may receive data packages from a design terminal through a standardized data interface. In some embodiments, the data packages contain the production parameters for the designed overflow brick, such as geometric dimensions (e.g., the overflow trough wall thickness B, the first height h₁ at the inlet cross-section, the second height h₂ at the inlet cross-section, and the support surface length l of the designed overflow brick) and material properties (e.g., the creep stress exponent n and the material density ρ_{y} of the overflow brick).

In some embodiments, after receiving the data packages containing the production parameters, the MES may automatically parse the data, verify the completeness and rationality of the production parameters, and accordingly generate the 3D digital model, such as a Computer-Aided Design (CAD) model.

The automated production unit is an integrated manufacturing facility. In some embodiments, the automated production unit may include equipment related to the fabrication of the designed overflow brick, such as raw material preparation equipment, green body forming equipment, high-temperature sintering equipment, and precision machining and inspection equipment.

In some embodiments, the raw material preparation equipment may include an automatic weighing and batching system, a high-energy ball mill or V-blender for ensuring uniform mixing of raw materials, and a spray drying tower for transforming mixed powder into granules suitable for pressing.

In some embodiments, the green body forming equipment may include a Cold Isostatic Press (CIP) for applying a uniform pressure to the powder to form a high-density green body, as well as ancillary systems such as an automatic feeding device matched with the CIP, and a robotic arm for grasping and transferring the green body.

In some embodiments, the high-temperature sintering equipment may include a high-temperature sintering kiln capable of precisely executing predefined temperature profiles. For example, the kiln may be an electric furnace or an atmosphere sintering furnace, equipped with an Automated Guided Vehicle (AGV) or a high-temperature resistant conveyor system for automatically loading and unloading the brick body.

In some embodiments, the precision machining and inspection equipment may include a multi-axis Computer Numerical Control (CNC) machining center or a CNC grinding machine for performing high-precision milling and grinding on the hardened brick body after sintering.

In some embodiments, the automated production unit may automatically complete the weighing and mixing of raw materials according to a material ratio specified by the production parameters, and press the mixed materials into a green body with a specified shape using an isostatic pressing technology. In some embodiments, the automated production unit may also feed the green body into the sintering kiln for high-temperature sintering under a strictly controlled temperature profile, causing the green body to densify and acquire a required physical strength.

In some embodiments, to ensure the green body achieves full densification during high-temperature sintering and to avoid cracking or deformation caused by thermal stress, the MES may match the green body with an optimal temperature profile. In some embodiments, the optimal temperature profile may be retrieved from a process knowledge base. For example, the MES may extract the geometric dimensions and material properties of a current designed overflow brick to be sintered as input parameters. Based on the input parameters, the MES may perform a query and match within the process knowledge base. In some embodiments, the process knowledge base may be structured in the form of a lookup table, where different combinations of geometric dimensions and material properties map to a unique, pre-defined, and experimentally validated temperature profile. In some embodiments, the process knowledge base may be constructed based on manual experience or historical data.

In some embodiments, the automated production unit may perform CNC precision machining on a sintered brick body of the designed overflow brick. For example, based on the 3D digital model, the automated production unit may use a CNC machine tool with diamond tools to mill and grind key forming surfaces of the sintered brick body, such as the overflow trough, the overflow inclined surfaces, and the brick tip chamfer, ensuring the final geometric accuracy of the designed overflow brick fully matches the acquired production parameters.

The forming apparatus refers to specialized equipment used in the glass substrate manufacturing process to form molten glass into flat glass of a specific specification. For example, the forming apparatus may be a fusion overflow forming system based on an overflow down-draw technique. It may be understood that the forming apparatus is used for manufacturing glass substrates by utilizing the designed overflow brick, not for fabricating the designed overflow brick itself.

In some embodiments of the present disclosure, by seamlessly integrating the acquired production parameters (e.g., the design parameters and the overflow trough wall thickness B of the designed overflow brick, etc.) with the automated manufacturing workflow, the high-strength and low-creep properties defined in the theoretical design are accurately realized. By directly converting the acquired production parameters into production instructions via the MES and utilizing automated units such as CNC precision machining, micrometer-level geometric control over key forming surfaces, including the overflow trough and the brick tip chamfer, is achieved. The high precision directly ensures the flowing stability of the molten glass, thereby significantly improving the thickness uniformity of the final glass substrate and the consistency and reliability between product batches.

The content provided above is intended only to illustrate the technical concepts of the present disclosure and should not be used to limit its scope of protection. Any modifications made to the technical solutions based on the technical ideas presented in the present disclosure fall within the scope of protection defined by the claims of the present disclosure.

1. A method for designing a high-strength low-creep overflow brick, comprising:
selecting a mature overflow brick for manufacturing a glass substrate as a reference overflow brick, obtaining design parameters of the reference overflow brick, and determining design parameters of a designed overflow brick;
determining a proximal leg height H_{Iref} and a distal leg height H_{Oref} of the reference overflow brick based on the design parameters of the reference overflow brick;
determining a first average leg height H_{1ref} and a second average leg height H_{2ref} of the reference overflow brick based on the design parameters, the proximal leg height H_{Iref}, and the distal leg height H_{Oref} of the reference overflow brick;
establishing similarity relationships between the reference overflow brick and the designed overflow brick, the similarity relationships including an overflow trough wall thickness similarity relationship, a first average leg height similarity relationship, and a second average leg height similarity relationship;
determining an overflow trough wall thickness B of the designed overflow brick based on the overflow trough wall thickness similarity relationship;
establishing a system of equations regarding a first height h₁ and a second height h₂ at an inlet cross-section of the designed overflow brick based on the design parameters, the proximal leg height H_{Iref}, the distal leg height H_{Oref}, the first average leg height H_{1ref}, and the second average leg height H_{2ref} of the reference overflow brick, the design parameters and the overflow trough wall thickness B of the designed overflow brick, and the similarity relationships between the reference overflow brick and the designed overflow brick, and determining the first height h₁ and the second height h₂ at the inlet cross-section of the designed overflow brick based on the system of equations;
determining strength parameters of the reference overflow brick and the designed overflow brick based on the design parameters, the proximal leg height H_{Iref}, and the distal leg height H_{Oref} of the reference overflow brick, and the design parameters, the overflow trough wall thickness B, and the first height h₁ and the second height h₂ at the inlet cross-section of the designed overflow brick; and
determining a support surface length l of the designed overflow brick and a maximum cylinder pressure Pₘₐₓ of a clamp cylinder based on the strength parameters of the reference overflow brick and the designed overflow brick.

2. The method for designing the high-strength low-creep overflow brick of claim 1, wherein the proximal leg height H_{Iref} of the reference overflow brick is determined based on Equation (1):
$${H}_{Iref} = {h}_{1 ref} - [ \frac{{w}_{ref} + 2 \times {B}_{ref}}{2 \times tan \frac{{θ}_{ref}}{2}} - ( \frac{{R}_{ref}}{sin \frac{{θ}_{ref}}{2}} - {R}_{ref} ) - {h}_{2 ref} ] , \qquad ( 1 )$$
and
the distal leg height H_{Oref} of the reference overflow brick is determined based on Equation (2):
$${H}_{Oref} = {H}_{Iref} + {h}_{ref} - {W}_{ref} \times tan {ϕ}_{ref} \qquad ( 2 )$$
in Equations (1) and (2), h_{1ref} denotes a first height at an inlet cross-section of the reference overflow brick, W_{ref} denotes an overflow surface width of the reference overflow brick, B_{ref} denotes an overflow trough wall thickness of the reference overflow brick, θ_{ref} denotes an overflow inclined surface angle of the reference overflow brick, R_{ref} denotes an overflow brick tip chamfer radius of the reference overflow brick, h_{2ref} denotes a second height at the inlet cross-section of the reference overflow brick, φ_{ref} denotes an overflow weir inclination angle of the reference overflow brick, and h_{ref} denotes an overflow trough depth at the inlet cross-section of the reference overflow brick.

3. The method for designing the high-strength low-creep overflow brick of claim 2, wherein the first average leg height H_{1ref} of the reference overflow brick is determined based on Equation (3):
$${H}_{1 ref} = \frac{{H}_{Iref} + {H}_{Oref}}{2} , \qquad ( 3 )$$
and
the second average leg height H_{2ref} of the reference overflow brick is determine based on Equation (4):
$${H}_{2 ref} = {H}_{1 ref} + ( {h}_{1 ref} - {H}_{Iref} ) . \qquad ( 4 )$$

4. The method for designing the high-strength low-creep overflow brick of claim 3, wherein
the overflow trough wall thickness similarity relationship is represented by Equation (5):
$$\frac{B}{{B}_{ref}} = {( \frac{W}{{W}_{ref}} )}^{\frac{n + 1}{n}} \times {( \frac{{h}_{ref}}{h} )}^{2} \qquad ( 5 )$$
the first average leg height similarity relationship is represented by Equation (6):
$$\frac{{H}_{1}}{{H}_{1 ref}} = \sqrt{{( \frac{W}{{W}_{ref}} )}^{\frac{n + 1}{n}} \times \frac{{w}_{ref} + 2 \times {B}_{ref}}{w + 2 \times B}} \qquad ( 6 )$$
the second average leg height similarity relationship is represented by Equation (7):
$$\frac{{H}_{2}}{{H}_{2 ref}} = \sqrt{{( \frac{W}{{W}_{ref}} )}^{\frac{n + 1}{n}} \times \frac{{W}_{2 ref}}{{W}_{2}}} \qquad ( 7 )$$
in Equations (5), (6), and (7), B denotes the overflow trough wall thickness of the designed overflow brick, B_{ref} denotes the overflow trough wall thickness of the reference overflow brick, H₁ denotes a first average leg height of the designed overflow brick, H₂ denotes a second average leg height of the designed overflow brick, W₂ denotes a leg width corresponding to the second average leg height H₂ of the designed overflow brick, W_{2ref} denotes a leg width corresponding to the second average leg height H_{2ref} of the reference overflow brick, n denotes a creep stress exponent of the designed overflow brick and the reference overflow brick, W denotes an overflow surface width of the designed overflow brick, W_{ref} denotes the overflow surface width of the reference overflow brick, h_{ref} denotes the overflow trough depth at the inlet cross-section of the reference overflow brick, and h denotes an overflow trough depth at the inlet cross-section of the designed overflow brick.

5. The method for designing the high-strength low-creep overflow brick of claim 4, wherein the overflow trough wall thickness B of the designed overflow brick is determine based on Equation (8):
$$B = {( \frac{W}{{W}_{ref}} )}^{\frac{n + 1}{n}} \times {( \frac{{h}_{ref}}{h} )}^{2} \times {B}_{ref} . \qquad ( 8 )$$

6. The method for designing the high-strength low-creep overflow brick of claim 4, wherein the system of equations regarding the first height h₁ and the second height h₂ at the inlet cross-section of the designed overflow brick includes:
Equation (9) regarding the proximal leg height H_{I} of the designed overflow brick:
$${H}_{I} = {h}_{1} - [ \frac{w + 2 \times B}{2 \times tan \frac{θ}{2}} - ( \frac{R}{sin \frac{θ}{2}} - R ) - {h}_{2} ] \qquad ( 9 )$$
Equation (10) regarding the distal leg height H₀ of the designed overflow brick:
$${H}_{0} = {H}_{I} + h - W \times tan ϕ \qquad ( 10 )$$
Equation (11) regarding the first average leg height H₁ of the designed overflow brick:
$${H}_{1} = \frac{{H}_{I} + {H}_{0}}{2} \qquad ( 11 )$$
Equation (12) regarding the second average leg height H₂ of the designed overflow brick:
$${H}_{2} = {H}_{1} + ( {h}_{1} - {H}_{I} ) \qquad ( 12 )$$
Equation (13) regarding the leg width w₂ corresponding to the second average leg height H₂ of the designed overflow brick:
$${w}_{2} = ( w + 2 \times B ) - \frac{1}{{H}_{2}} \times {( {H}_{2} - {H}_{1} )}^{2} \times tan \frac{θ}{2} \qquad ( 13 )$$
Equation (14) regarding the first average leg height H₁ of the designed overflow brick, which is determined based on the first average leg height similarity relationship:
$${H}_{1} = {H}_{1 ref} \times \sqrt{{( \frac{W}{{W}_{ref}} )}^{\frac{n + 1}{n}} \times \frac{{w}_{ref} + 2 \times {B}_{ref}}{w + 2 \times B}} \qquad ( 14 )$$
Equation (15) regarding the second average leg height H₂ of the designed overflow brick, which is determined based on the second average leg height similarity relationship:
$${H}_{2} = {H}_{2 ref} \times \sqrt{{( \frac{W}{{W}_{ref}} )}^{\frac{n + 1}{n}} \times \frac{{W}_{2 ref}}{{W}_{2}}} \qquad ( 15 )$$
in Equation (9), R denotes an overflow brick tip chamfer radius of the designed overflow brick, θ denotes an overflow inclined surface angle of the designed overflow brick, and φ denotes an overflow weir inclination angle of the designed overflow brick.

7. The method for designing the high-strength low-creep overflow brick of claim 6, wherein the strength parameters include an equivalent thickness, an equivalent height base value, and an equivalent height, and the determining strength parameters of the reference overflow brick and the designed overflow brick includes:
determining an equivalent thickness of the reference overflow brick and an equivalent thickness of the designed overflow brick based on Equation (16) and Equation (17), respectively:
$$b = ( w + 2 \times B ) - 2 \times ( {h}_{1} - {H}_{I} ) \times tan \frac{θ}{2} \qquad ( 16 )$$
$${b}_{ref} = ( {w}_{ref} + 2 \times {B}_{ref} ) - 2 \times ( {h}_{1 ref} - {H}_{Iref} ) \times tan \frac{{θ}_{ref}}{2} \qquad ( 17 )$$
where, b_{ref} denotes the equivalent thickness of the reference overflow brick, and b denotes the equivalent thickness of the designed overflow brick;
determining an equivalent height base value of the reference overflow brick and an equivalent height base value of the designed overflow brick based on Equation (18) and Equation (19), respectively:
$${d}^{′} = \frac{1}{2 \times b} \times ( w + 2 B ) \times ( h + {h}_{1} + {h}_{2} + {H}_{0} ) \qquad ( 18 )$$
$${{d}^{′}}_{ref} = \frac{1}{2 \times {b}_{ref}} \times ( {w}_{ref} + 2 {B}_{ref} ) \times ( {h}_{ref} + {h}_{1 ref} + {h}_{2 ref} + {H}_{0 ref} ) \qquad ( 19 )$$
where, d′_{ref} denotes the equivalent height base value of the reference overflow brick, and d′ denotes the equivalent height base value of the designed overflow brick; and
determining an equivalent height d of the designed overflow brick and an equivalent height d_{ref} of the reference overflow brick based on Equation (20) and Equation (21), respectively:
$$d = {d}^{′} \times ( - 1.718936 \times {10}^{- 5} \times ( b \times {d}^{′} \times W \times {ρ}_{y} ) + 1.169457 ) \qquad ( 20 )$$
$${d}_{ref} = {d}_{ref}^{′} \times ( - 1.718936 \times {10}^{- 5} \times ( {b}_{ref} \times {d}_{ref}^{′} \times {W}_{ref} \times {ρ}_{y} ) + 1.169457 \qquad ( 21 )$$
where, b denotes the equivalent thickness of the designed overflow brick, b_{ref} denotes the equivalent thickness of the reference overflow brick, d′ denotes the equivalent height base value of the designed overflow brick, d′_{ref} denotes the equivalent height base value of the reference overflow brick, d denotes the equivalent height of the designed overflow brick, d_{ref} denotes the equivalent height of the reference overflow brick, H_{Oref} denotes the distal leg height of the reference overflow brick, and ρ_{y} denotes a material density of the designed overflow brick and the reference overflow brick.

8. The method for designing the high-strength low-creep overflow brick of claim 7, wherein the support surface length l of the designed overflow brick is determined based on Equation (22):
$$l = {( \frac{d}{{d}_{ref}} )}^{2} \times \frac{{W}_{ref}}{W} \times {l}_{ref} \qquad ( 22 )$$
where, l_{ref} denotes a support surface length of the reference overflow brick.

9. The method for designing the high-strength low-creep overflow brick of claim 7, wherein the maximum cylinder pressure Pₘₐₓ of the clamp cylinder for the designed overflow brick is determined based on Equation (23):
$${P}_{max} = 1.251 \times W \times b \times d \times {p}_{y} \qquad ( 23 )$$
where, d denotes the equivalent height of the designed overflow brick, b denotes the equivalent thickness of the designed overflow brick, and W denotes the overflow surface width of the designed overflow brick.

10. (canceled)
